(19) United States
(12) Patent Application Publication
(10) Pub. No.: US 2014/0273249 A1
(43) Pub. Date: Sep. 18, 2014

(54) High Range Activated Clotting Time Assay Formulation

(71) Applicant: MEDTRONIC, INC., MINNEAPOLIS, MN (US)

(72) Inventors: Charlene Yuan, Woodbury, MN (US); Trevor C. Huang, Maple Grove, MN (US); Tessy Kanayinkal, Brooklyn Park, MN (US); Mary Jo Passenheim, Coon Parids, MN (US); Craig D. Petersen, Brooklyn Park, MN (US); Ivan Akunovich, Brooklyn Park, MN (US)

(73) Assignee: MEDTRONIC, INC., Minneapolis, MN (US)

(21) Appl. No.: 13/795,608

(22) Filed: Mar. 12, 2013

(51) Int. Cl. G01N 33/86

(52) U.S. Cl. CPC G01N 33/86
USPC 436/69; 422/73; 427/337; 427/230; 427/569

(57) ABSTRACT

High range activated clotting time (HR-ACT) tests detect blood clotting time in blood samples which have high levels of heparin. Reagents such as calcium chloride and kaolin within the test apparatus trigger clotting. The cartridge is treated with a strong surface treatment process, such as an atmospheric plasma treatment, to increase the hydrophilic property of the test chamber, there may be a significant reduction in the kaolin concentration required to activate the blood sample and initiate the coagulation process. The kaolin concentration may be further reduced if the buffer component used in the buffer saline contains phosphate. The reduction of the kaolin concentration allows more calcium to be released from the kaolin to participate in the clotting process. The combined effect of adding a surface treatment to the cartridge to increase the hydrophilic property of reaction chamber and adding phosphate into buffered saline allows for clot detection of blood samples containing 5˜6 U/mL heparin.

### TECHNICAL FIELD

This invention relates to detecting changes in viscosity of biologic fluid test samples, e.g., detecting coagulation and coagulation-related activities including agglutination and fibrinolysis of human blood test samples, and more particularly to improved methods and apparatus for obtaining a coagulation time of a blood test sample.

### BACKGROUND

Blood coagulation is a complex chemical and physical reaction that occurs when blood comes into contact with an activating agent, such as an activating surface or an activating agent. (In this context, the term “blood” means whole blood, citrated blood, platelet concentrate, plasma, or control mixtures of plasma and blood cells, unless otherwise specifically called out otherwise; the term particularly includes heparinized blood.)

Several tests of coagulation are routinely utilized to assess the complicated cascade of events leading to blood clot formation and test for the presence of abnormalities or inhibitors of this process. Among these tests are activated clotting time (ACT), which includes high range ACT (HRACT), a test which features a slope response to moderate to high heparin levels in whole blood drawn from a patient during cardiac surgery.

During heart bypass surgery, real-time assessment of clotting function at the operative site is performed to evaluate the result of therapeutic interventions and also to test and optimize, a priori, the treatment choice and dosage.

High Range Activated Clotting Time (HR-ACT) is a test used to monitor the effect of high levels of heparin (up to 6 U/ml) during cardiac pulmonary bypass surgery. HR-ACT tests are based on the viscosity change of a test sample within a test chamber. During a test cycle, a ferromagnetic washer immersed in the test sample is lifted to the top of the test chamber by magnetic force produced by a magnetic field located at the top of the test chamber; the washer is then held at the top of the test chamber for a specific time. After the specified holding time, the washer is then dropped through the test sample via gravity. The increased viscosity due to the clotting of the test sample of blood clotting slows the motion of the washer. Thus, if the time that the washer travels through a specified distance (i.e., the washer “drop time”) is greater than a preset value (the clot detection sensitivity threshold), a clot is detected and an HR-ACT value is reported.

A particular apparatus and method for detecting changes in human blood viscosity based on this principle is disclosed in U.S. Pat. Nos. 5,629,209 and 6,613,286, in which heparinized blood is introduced into a test cartridge through an injection port and fills a blood receiving/dispensing reservoir. The blood then moves from the reservoir through at least one conduit into at least one blood-receiving chamber where it is subjected to a viscosity test. A freely movable ferromagnetic washer is also located within the blood-receiving chamber that is moved up using an electromagnet of the test apparatus and allowed to drop with the force of gravity. Changes in the viscosity of the blood that the ferromagnetic washer falls through are detected by determining the position of the ferromagnetic washer in the blood-receiving chamber over a given time period or a given number of rises and falls of the ferromagnetic washer. The blood sample can be mixed with a viscosity-altering agent (e.g., protamine) as it passes through the conduit to the blood-receiving chamber. Air in the conduit and blood-receiving chamber is vented to atmosphere through a further vent conduit and an air vent/fluid plug as the blood sample is fills the blood-receiving chamber.

The movement of the washer in the above approach is actively controlled only when it is moved up, and the washer passively drops with the force of gravity. The washer is free to float in the test chamber and may drift side-to-side as it is moved up or floats downward. The side-to-side drifting movement may affect the rise time and the fall time, which could add error to the coagulation time measured. The washer may eventually stop moving as a clot forms about it, and no additional information can be obtained on the coagulation process in the sample.

### SUMMARY

It has been discovered that, in a blood sample that is heparinized with high level of heparin, the anticoagulant effect of the heparin requires a higher level of calcium to promote clotting than in conventional tests at lower heparin levels. Conventional tests involve a contact activator, or a mixture of contact activators, such as kaolin, celite and glass beads in a buffered saline solution. Calcium chloride is mixed with the buffered activator suspension solution. The activation reagent is dispensed into the test chamber and then dried (in the dry reagent format). The discovery that the dried kaolin and calcium chloride mixture does not release all the calcium back to the solution after it is mixed with test fluid cannot be addressed by simply increasing the calcium concentration in the calcium-kaolin mixture. An optimal kaolin-calcium ratio in the HR-ACT formulation is critical for reliable activated clotting time measurements in the presence of high levels of heparin (5 to 6 U/mL). When the calcium chloride concentration in the mixture is too high, the dried calcium chloride is a hygroscopic agent; it competes with kaolin for water molecules. As a result, high calcium chloride concentrations may cause aggregation of dry kaolin (or, a “caking” effect), and reduce the amount of kaolin surface available for clotting factors to bind, thus prolonging clotting time. By contrast, when the calcium chloride concentration is too low, the calcium ion is not all freed from the kaolin to bind to clotting factors, or to inhibit the anticoagulant effect of the heparin, and thus the dry formulation of kaolin mixed with calcium cannot enable blood samples to clot in the presence of high levels of heparin (5 to 6 U/ml). Thus, the calcium concentration must be kept within strict limits.

While one approach to this problem is physical separation of calcium chloride from the kaolin suspension solution, that approach introduces additional steps into the manufacturing of the cartridges and thus additional costs, quality control issues, and the like. In addition, to achieve the desired goal of clot detection in blood samples containing 5-6 U/ml of heparin, it is generally necessary to modify the chemical composition of the kaolin and calcium chloride suspensions to adapt them to this approach. That also introduces undesirable costs for manufacturing and quality control.

By contrast, it has been discovered that if a cartridge is treated with a strong surface treatment process, such as an atmospheric plasma treatment, to increase the hydrophilic property of the test chamber, there may be a significant reduction in the kaolin concentration required to activate the blood sample and initiate the coagulation process. The kaolin concentration may be further reduced if the buffer component used in the buffer saline contains phosphate. The reduction of the kaolin concentration may allow more calcium to be released to participate in the clotting process. The combined effect of adding a surface treatment to the cartridge to increase the hydrophilic property of the reaction chamber and adding phosphate into buffered saline allows for clot detection of blood samples containing 5˜6 U/mL heparin. With this embodiment, the kaolin concentration is significantly reduced and the physical separation of calcium from the kaolin reagent is not required.

The surface treatment of the cartridge also promotes even spreading of the kaolin reagent into the cartridge test chamber during the reagent dispense process, forming a visibly smooth kaolin surface after drying. Even distribution of the kaolin reagent in the test chamber greatly improves the function of the HR-ACT test; it minimizes air pockets formed on the uneven kaolin surface in the test chamber during sample injection, and it also improves kaolin suspension during sample mixing, as confirmed by observations of air bubbles released from the kaolin.

The surface treatment of the cartridge also cleans the cartridge. It has been discovered that the outgassing of the cartridge plastic material deposits chemicals onto the washer surfaces during cartridge storage. As a result of cartridge outgassing, the hydrophilic surface of the washer deteriorates over time and reduces the cartridge shelf life. Surface treatment significantly reduces the volatile chemicals from the cartridge and increases cartridge shelf life.

Dry kaolin re-suspension is further improved by adding into the wet kaolin/reagent mixture a zwitterion surfactant such as HEPES (4-(2-hydroxyethyl)-1-piperazineethanesulfonic acid) or a similar compound, or a non-ionic surfactant and emulusifier such as polysorbate-80.

To increase the speed of the kaolin drying process, it has been discovered that methanol can be added to the wet kaolin/reagent mixture. After drying, the methanol is evaporated from the kaolin mixture and does not interfere with the blood clotting process.

Thus, in general terms, in one embodiment, an improved cartridge for blood clot detection comprises a test chamber with a strong hydrophilic surface. The hydrophilic surface reduces the amount of negatively charged reagent required in the test chamber, and allows the positively charged reagent to be released from a mixture of it and the negatively charged reagent. Physical separation of the positively charged reagent and negatively charged reagent within the chamber is not required. Thus, the positively charged and negatively charged reagents may be combined into a “modified reagent” mixture. The positively charged reagent in this mixture may comprise calcium or, independently, the negatively charged reagent in this mixture may comprise kaolin.

The surface treatment may be an atmospheric plasma treatment, but it need not be. Other alternatives include any process that increases the surface energy of the cartridge by an amount sufficient to achieve a water contact angle of less than about 60 degrees, or more specifically between about 60 and about 20 degrees, as measured by conventional techniques (e.g., the static sessile drop method). As long as the requisite water contact angle is achieved, the process may be any alternative to atmospheric plasma treatment, although as the person of ordinary skill in the art would appreciate, the alternatives may provide different cost and/or performance tradeoffs. In general, it is believed that the alternative treatments would likely be cost prohibitive or inferior in performance (or both) at this time, but that does not foreclose their use from a technological standpoint.

The negatively charged reagent can be further reduced by using a buffering agent such as phosphate containing buffer.

This summary of the claims has been presented here simply to point out some of the ways that the claims overcomes difficulties presented in the prior art and to distinguish the claims from the prior art and is not intended to operate in any manner as a limitation on the interpretation of claims that are presented initially in the patent application and that are ultimately granted.

### BRIEF DESCRIPTION OF THE DRAWINGS

These and other advantages and features will be more readily understood from the following detailed description of various embodiments, when considered in conjunction with the drawings, in which like reference numerals indicate identical structures throughout the several views, and in which:

FIG. 1, which is based on FIG. 13 of U.S. Pat. No. 5,629,209, is a cross-sectional view of a cartridge positioned within a machine.

FIG. 2, which is based on FIG. 12d of U.S. Pat. No. 5,629,209, is a partial cross-sectional view of the cartridge of FIG. 1.

FIG. 3 is a schematic cross-section of a first embodiment of the test chamber portion of the cartridge of FIGS. 1 and 2.

FIG. 4 is a graph of data of ACT response to heparin in the embodiment of FIG. 3.

### DETAILED DESCRIPTION

In the following detailed description, references are made to illustrative embodiments of methods and apparatus for carrying out the claims. It is understood that other embodiments can be utilized without departing from the scope of the claims. Exemplary methods and apparatus are described for performing blood coagulation tests of the type described above.

FIG. 1 only illustrates the basic features of a suitable apparatus, as known from U.S. Pat. No. 5,629,209, the entirety of which is incorporated by reference. The cartridge 100, having been inserted into the side 16 of the machine 10, is secured within the cartridge holder 302. An aperture 28 enables the fluid sample to be introduced into the cartridge 100 after the cartridge 100 is inserted into the machine 10. An air vent/fluid plug device 120 is aligned over a hole 304 in the base of the cartridge holder 302 to permit escape of air that is vented from the cartridge 100 during the movement of the fluid sample into its respective fluid-receiving chamber. Each fluid-receiving chamber may be associated with a means for moving the ferromagnetic material (e.g., a washer made of a ferromagnetic material) provided by the machine 10, such as an electromagnet 122, and a means for detecting the position of the ferromagnetic material 116 within the chamber 114, e.g., a detector 124. A radio frequency detector may be conveniently employed for this purpose. It should be noted that the detector 124 is not limited to the detection of ferromagnetic material but is capable of detecting any metallic substance placed within the chamber 114. The electromagnet 122 and the position detector 124 are connected to a circuit board 300 through which an associated computer receives information, provides directions, and provides test results. For simplicity of illustration, only one fluid-receiving chamber 114, electromagnet 122, and position detector 124 are shown. Cartridge 100 may have a plurality of such arrangements for alternative and/or comparative tests.

FIG. 2 illustrates that fluid 200 fills the fluid-receiving chamber and reaches the air vent/fluid plug device 120 to establish a fluid lock. Ferromagnetic washer 116 is moved between a resting position on the bottom of the fluid-receiving chamber 114 and the top of the chamber 114 as the electromagnet 122 is energized; if the electromagnet 122 is turned off the washer 116, under the force of gravity, falls through the fluid 200 to the bottom of the chamber 114. The position detector 124 measures the time required for the washer 116 to fall from the top to the bottom of the chamber 114 and sends this information to the associated computer. As the viscosity of the fluid 200 increases, the measured time increases. Indeed, in the case of blood coagulation, eventually, a washer 116 is unable to move through a blood sample.

When the fluid 200 whose viscosity is being measured is blood, the motion of the washer 116 through the blood also has the effect of activating the clotting process of the blood. The activation effect is enhanced when the surface of the washer 116 is roughened in known ways, as such techniques increase the surface area of the washer. If even faster clotting times are necessary, a viscosity-altering substance may be used. For example, a clotting activator such as tissue thromboplastin can be added to the cartridge, or a particulate activator such as diatomaceous earth or kaolin may be used either alone or in combination with a viscosity-altering substance such as protamine or thromboplastin.

The position detector 124 may be a radio frequency detector. Radio frequency detectors sense the position of the washer 116 by sensing the changes in the magnetic field surrounding the detection coil of the radio frequency detector that are caused by the presence of the washer 116. Radio frequency detectors also are sensitive to ferromagnetic and other metallic materials and resistance to effects caused by other elements of the device, such as the fluid. It should be understood, however, that other types of position detectors 124 are contemplated. For example, in another embodiment, the position detector 124 is a Hall effect sensor and its associated circuitry, as generally described in U.S. Pat. No. 7,775,976 (the entirety of which is incorporated by reference) at column 16, line 15 to column 17, line 5. Regardless of the type of position detector 124 employed, the absolute position of the washer 116 is measured and used as described below.

In a typical sequence, a sample mix cycle begins the test protocol. The electromagnet 122 initially raises and lowers the washer 116 rapidly several times to further mix the fluid 200 with any viscosity-altering substance present and, if the fluid 200 is blood, promote activation of clotting, as discussed above. The fluid 200 is then allowed to rest for a short time. During the subsequent test itself, the electromagnet 122 raises the washer 116 repeatedly at a slower rate. After each elevation of the washer, the position detector 124 is used to determine the “fall time” (or “drop time”), i.e., the time taken for the washer 116 to fall to the bottom of the chamber 114. Absence of an increase in fall time suggests a lack of coagulation and the test continues. But an increase in fall time suggests a change in viscosity, measured in terms of the amount of fall time as compared to a baseline value. All data, including individual test results, may be displayed, stored in memory, printed, or sent to another computer, or any combination of the same.

The principles of the first embodiment are schematically illustrated in FIG. 3. The electromagnet 122, position detector 124, and fluid 200 have been omitted for clarity only. Similarly, the height of the chamber 114 is exaggerated relative to the thickness of the washer 116 only for purposes of illustration.

The material selected for cartridge 100 may be any medical grade material having suitable properties, such as commercially available injection moldable resins. Examples include polycyclohexylendimethylene terephthalate glycol (PCTG), polycarbonates, and acrylics having comparable properties. Blends of such materials are also suitable provided other design requirements are met.

Regardless of material, entrapped air within the cartridge or assay causes uncontrolled coagulation and inaccurate reagent concentration, both of which are contrary to the design objective of the system as a whole. Surfaces of high wetability will cause the blood sample to more readily displace air out of the assay during a filling cycle, and undesirable thrombogenic effects may begin to occur. If the wettability is too low, the surface does not sufficiently eliminate air.

Balancing these two factors suggests a surface which inherently has or is treated to have a water contact angle between about 60 degrees (e.g., in the range of 50-60 degrees) and about 20 degrees (e.g., in the range of 20-25 degrees) for an extended duration, such as for at least six months.

Untreated cartridges formed from PCTG may have a water contact angle of 70 degrees or more, and untreated polycarbonates may have a water contact angle approaching 90 degrees. Thus, for those materials and others, surface treatment to achieve the desired water contact angle is indicated, and in such cases it is desirable to have a surface treatment process or design which minimizes any propensity to entrap air. The surface treatments described here for PCTGs are particularly desirable for that material, because PCTGs are known to be inherently neutral to wetting.

In general terms, surface energy treatments are suitable for this application if they increase the adhesion or wettability properties between the dissimilar materials of the cartridge and the reagent. Among different processes used to achieve these ends, the method of bombarding ionized gas (the plasma state) onto the cartridge surface can be used, a process more generally referred to “plasma treatment.” This process has two effects; first, it functionalizes the surface, meaning functional groups (ionized gas molecules) are grafted onto the material surface, and second, it cleans the surface by burning off oil residues or other organic compounds that might be present (such as those commonly found in resin additives, in the case of plastics used to form the cartridge).

For example, other methods of achieving higher surface energy recognized in industry include (but are not limited to) use of chemical coatings, resin additives, or even a different “flavor” or medium of plasma, i.e., pure argon, pure nitrogen, pure oxygen, or some mixture of any of these or other gases. Other alternatives include plasma enhanced vapor deposition (PEVD), by which the plasma medium includes trace amounts of vaporized polymers that permanently deposit a layer a few molecules thick on the surface of the cartridge. Other alternative treatments include non-atmospheric plasma treatments, either higher than atmospheric pressure or lower than atmospheric pressure; such techniques typically require the treatment equipment to be gas-tight for batch processing. Another type of alternative available in the selection of the surface treatment is the selection of the manner in which the gas is ionized to produce the plasma, such as by voltage discharge (arcing) or RF energy as known in the art. With any of these variations above, as long as the requisite water contact angle is achieved, the process may be used in alternatives to the embodiment of atmospheric plasma treatment, although as the person of ordinary skill in the art would appreciate, the alternatives may provide different cost and/or performance tradeoffs. In general, it is believed that the alternative treatments would likely be cost prohibitive or inferior in performance (or both) at this time, but that does not foreclose their use from a technological standpoint.

Specifically, at least one interior surface of fluid-receiving chamber 114 is treated with a surface treatment technique, such as the atmospheric plasma treatment described above. As illustrated by way of non-limiting example, the bottom surface 134 is so treated (although of course, other surfaces or the entire interior of chamber 114 may be so treated). After treatment, the fluid-receiving chamber 114 is hydrophilic; thus less kaolin (if that is the reagent chosen) is required to initiate clotting. When kaolin is used, it may be further reduced using a buffering agent such as phosphate buffer saline solution to form modified composition 250. Thus, the modified composition 250 is coated onto a surface treated portion of the test chamber. In the embodiment illustrated, this is the bottom of the chamber as illustrated, but in general terms it could be other surfaces up to an including the entire interior surface of the test chamber, even if the composition is coated only onto a portion of the interior.

A suitable surface treatment process is provided by an atmospheric plasma treatment apparatus commercially available from PlasmaTreat as model FG1001. The plasma media is ambient pressure atmospheric gas (oil free) at less than 20% relative humidity, filtered sufficiently to ensure that no particulates over 0.3 micron in size are present. Default parameters of 280V, plasma power pulse frequency of 21.0 KHz, and inlet air pressure of 3 Barr (as measured at the regulator on the transformer) are suitable. A nozzle size providing a 1 inch diameter treatment area at a rotation speed of 2800 RPM is effective. A feed rate of 100 mm/sec and gap between the nozzle tip and processing surface of 8.0 mm is sufficient to treat the surface of the cartridge in two passes.

When the cartridge 100 is used in testing, the blood specimen will dissolve the modified composition (typically calcium chloride and kaolin) 250 which will activate the blood specimen and initiate the clotting process.

The schematically-illustrated height of modified composition 250 in FIG. 3 is exaggerated solely for clarity. With the combination of both the surface treatment and the buffer agent treatment, detection of a clot in a blood sample having a high level of heparin may be achieved despite combination of the calcium chloride into modified kaolin composition 250.

The surface treatment of the fluid-receiving chamber 114 allows for an even spread of the modified composition 250, to form a smooth surface 251 after the modified composition 250 dries. The surface smoothness of the dry kaolin composition 250 will minimize the formation of air pockets during the blood sample injection. Large pockets of air trapped in the test chamber 114 hinder the free movement of washer 116 and can cause a test failure. Small pockets of air interfere with the re-suspension of the dry kaolin during a sample mixing cycle, and can provide erroneous clotting time results. Another advantage of the surface treatment is that it also cleans the fluid-receiving chamber 114, which reduces the deposition of the outgassing volatile chemicals from the plastics of the fluid-receiving chamber 114 onto the washer 116. The outgassing chemical deposition on washer 116 reduces the hydrophilic property of the washer 116 during cartridge storage, reduces the shelf life of the cartridge.

To promote re-suspension of the dry kaolin when it is used in modified composition 250, a zwitterion surfactant or a non-ionic surfactant and emulusifier, such as polysorbate-80, may be added. Another possible function of the surfactant is to reduce the volume of any air pocket which may form on the surface of modified composition 250 when it is in contact with the blood sample. In general, compositions within the following ranges are acceptable, although interactions between these components must also be considered: kaolin in the range of 0.70% to 2.53%; 5 to 15 mM calcium chloride (CaCl₂), and 0.035% to 0.07% polysorbate-80 (brandname “TWEEN 80”).

Methanol may also be added to the modified composition 250 to aid the drying process, particularly when kaolin is used in the composition. It has been discovered that methanol does not interfere with the clotting assay once it has evaporated during the drying process. One specific possible composition is: 3.75% kaolin in 37.5 mM calcium chloride (CaCl₂) and 0.185% polysorbate-80 (brandname “TWEEN 80”), combined with 40% phosphate buffered saline (PBS) and 50% methanol (CH₃OH or sometimes “MeOH”).

### Example 1

FIG. 4 shows a comparison of results from a cartridge made as described in U.S. Pat. No. 6,613,286. The graph is time to detect a clot (seconds) as a function of heparin concentration (U/ml). The plasma treated cartridge was coated with kaolin reagent mixed with phosphate buffer, calcium chloride, polysorbate-80 and methanol. Blood samples from three donors (denoted D134, D158, and D317) each heparinized with 2, 4 and 6 U/mL heparin were tested in this experiment. All three donors detected clot time at 6 U/mL heparin levels.

While the description above uses the apparatus and procedures of U.S. Pat. Nos. 5,629,209 and 6,613,286 to describe certain details, the broadest scope of the disclosure includes any apparatus which relies on any combination of analog or digital hardware, as well as methods of manufacturing or using the same, that do not depend upon the specific physical components mentioned above but nonetheless achieve the same or equivalent results. Therefore, the full scope of the invention is described by the following claims.

What is claimed is:

1. A cartridge for blood clot detection, comprising a test chamber having an interior, at least a portion of the interior having a hydrophilic surface upon which lies a composition comprising a first positively charged reagent reduced with a buffering agent and a second, negatively charged reagent.

2. The cartridge of claim 1, in which the first reagent comprises calcium.

3. The cartridge of claim 1, in which the second reagent comprises kaolin.

4. The cartridge of claim 1, in which the composition comprises a buffer saline containing phosphate.

5. The cartridge of claim 1, in which composition comprises at least one of a zwitterion surfactant and a non-ionic surfactant and emulusifier.

6. A method of manufacturing a cartridge for measuring clotting time of a sample of blood introduced into a chamber within the cartridge, comprising forming the cartridge to have an interior hydrophilic surface upon which lies a composition comprising a first positively charged reagent reduced with a buffering agent and a second, negatively charged reagent.

7. The method of claim 6, in which the first reagent comprises calcium.

8. The method of claim 6, in which the second reagent comprises kaolin.

9. The method of claim 6, in which the composition comprises a buffer saline containing phosphate.

10. The method of claim 6, further comprising adding methanol to the composition and allowing the methanol to evaporate as the composition dries.

11. A method of detecting formation of a clot in a blood sample with a washer moving through the sample, comprising:
a. providing a cartridge defining a test chamber for the sample, the cartridge comprising the washer within the test chamber;
b. providing the cartridge with an interior in which at least a portion of the interior has a hydrophilic surface upon which lies a composition comprising first positively charged reagent reduced with a buffering agent and a second, negatively charged reagent; and
c. introducing the blood sample into the test chamber such that the composition of the first and second reagents is mixed into the blood sample.

12. The method of claim 11, in which the first reagent comprises calcium.

13. The method of claim 11, in which the second reagent comprises kaolin.

14. The cartridge of claim 11, in which the composition comprises a buffer saline containing phosphate.

15. A method of manufacturing a cartridge for measuring clotting time of a sample of blood introduced into a chamber within the cartridge, comprising providing the cartridge with an interior, treating at least a portion of the interior to have a water contact angle between about 20 and about 60 degrees, and providing onto the hydrophilic surface a composition comprising a first positively charged reagent reduced with a buffering agent and a second, negatively charged reagent.

16. The method of claim 15, in which treating the portion of the interior of the cartridge increases adhesion between dissimilar materials of the cartridge and the reagent.

17. The method of claim 15, in which treating the portion of the interior of the cartridge comprises atmospheric plasma treatment.

18. The method of claim 15, in which treating the portion of the interior of the cartridge comprises one of applying a chemical coating or resin additive.

19. The method of claim 15, in which treating the portion of the interior of the cartridge comprises applying plasma from one of argon, nitrogen, or oxygen.

20. The method of claim 15, in which treating the portion of the interior of the cartridge comprises applying plasma enhanced vapor deposition (PEVD).

21. The method of claim 15, in which treating the portion of the interior of the cartridge comprises applying a non-atmospheric plasma treatment which is either higher than atmospheric pressure or lower than atmospheric pressure.
